(19) United States
(12) Patent Application Publication
(10) Pub. No.: US 2009/0209881 A1
(43) Pub. Date: Aug. 20, 2009

(54) Apparatus for manually retaining a urine cup while providing a urine specimen

(76) Inventor: Carole A. Willcocks, Santa Monica, CA (US)

Correspondence Address: ROZSA LAW GROUP LC, 18757 BURBANK BOULEVARD, SUITE 220, TARZANA, CA, 91356-3346, US

(21) Appl. No.: 11/805,134

(22) Filed: May 22, 2007

(51) Int. Cl. A61B 5/00

(52) U.S. Cl. 600/573

(57) ABSTRACT

The present invention is a hand held apparatus to manually retain a urine specimen cup between the legs of a person so that a person can provide a urine specimen into the cup while precisely holding the cup beneath his or her legs so that the urine can be precisely voided into the cup.

### BACKGROUND OF THE INVENTION

1. Field of the Invention

The present invention relates to the field of providing a specimen of urine when a patient is in a doctor's office, hospital, or laboratory, or other location where a urine specimen from the patient is required.

2. Description of the Prior Art

In general, the concept of retaining a urine cup to provide a urine specimen is known.

The following 17 patents and published patent applications are the closest prior art references which were uncovered in the search. A complete set of copies of these patents and patent applications are enclosed herewith for your review.

1. U.S. Pat. No. 2,603,795 issued to Marion E. Terlizzi and assigned to George Billy on Jul. 22, 1952 for “Specimen Receptacle” (hereafter the “Terlizzi patent”);

2. U.S. Pat. No. 2,801,424 issued to John E. Mercer on Aug. 6, 1957 for “Urine Sampling Device” (hereafter the “Mercer patent”);

3. U.S. Design Pat. No. Des. 194,965 issued to Louis Guss on Apr. 2, 1963 for “Support For Urine Specimen Container Or Similar Article” (hereafter the “Guss Design patent”);

4. U.S. Pat. No. 3,466,145 issued to Charles M. Van Duyne on Sep. 9, 1969 for “Urine Screening Device” (hereafter the “Van Duyne patent”);

5. U.S. Pat. No. 4,137,573 issued to Daniel E. Kroeger on Feb. 6, 1979 for “Urine Specimen Collector” (hereafter the “Kroger patent”);

6. U.S. Pat. No. 5,146,637 issued to Mark Bressler et al. on Sep. 15, 1992 for “Female Urine Collection Apparatus” (hereafter the “Bessler patent”);

7. U.S. Pat. No. 5,671,485 issued to Robert E. Middlestead on Sep. 30, 1997 for “Support Device For Emptying Colostomy Bag” (hereafter the “Middlestead patent”);

8. U.S. Pat. No. 6,151,972 issued to Jacob Cloete Venter et al. and assigned to V&N Projects CC on Nov. 28, 2000 for “Urine Sampling Device” (hereafter the “Venter patent”);

9. U.S. Pat. No. 6,212,698 issued to Beverly J. Stingley et al. on Apr. 10, 2001 for “Urine Collection Kit” (hereafter the “Stingley patent”);

10. U.S. Pat. No. D489,453 issued to Rodney S. Sapyta on May 4, 2004 for “URINE SPECIMEN CUP HOLDER” (hereafter the “Sapyta patent”);

11. U.S. Pat. No. 6,811,754 issued to Cheme G. House on Nov. 2, 2004 for “Biological Specimen Collection Apparatus” (hereafter the “House patent”);

12. U.S. Pat. No. 7,011,634 issued to Robert W. Paasach et al. and

13. United States Published Patent Application No. 2006/0184064 issued to Robert W. Paasch et al. For “Urine Collection Sample Device (hereafter “Paasch Published Application”);

14. U.S. Pat. No. 6,775,852 issued To Alvarez et al. on Aug. 17, 2004 for “Urine Collection Device” (hereafter the “Alvarez patent”);

15. United States Published Patent Application 2006/0090253 to El Haje for “Urine Or Stool Sample Collector” (hereafter “El Haje Published Patent Application”);

16. United States Published Patent Application 2004/0241052 to House for “Biological Specimen Collection Apparatus And Method (hereafter “House Published Patent Application”); and

17. International Published Application WO 2006/001690 to Dijkman for “Device or Measuring Data Relating To Urine Production Of A Patient (hereafter “Dijkman Published Patent Application”).

The Terlizzi patent is a specimen receptacle which discloses the concept of having a specimen receptacle 18 which is screwed into and retained between the arms 20 and 24 which can thereafter be retained on a toilet bowl as shown in FIG. 1.

The Mercer patent discloses a urine sampling device. The urine sampling device is specifically for women. There is a pair of supporting members 12 and 13 which retain the urine sampling device between the bottom of a toilet bowl and held in position by the toilet seat. There is a cup holder 14 and a deflector holder 15. The urine impinging on a deflector 16 will flow into the sample cup 17 without the need for handling these members other than to place them in and remove them from the device.

The Guss Design patent is entitled a “Support For Urine Specimen Container” but it does not disclose the container itself as how it is supported other than from the side view of FIG. 3.

The Van Duyne patent is for a urine screening device. The device includes an elongated sheet structure embodying the central holder section 12 and tongue or upper support section 14 which is bent at substantially right angles to the holder section 12. There are also a pair of wings or splash shields 16a and 16b and a visual indicating strip 18. Urine is deposited in the cup and the strip is used to examine the urine.

The Kroeger patent is a urine specimen collector which can be attached to the front rim of a toilet bowl as shown in FIG. 1. The device which can removably receive a liner assembly 12 includes an annular receptacle portion 13 having an upper peripheral flange portion 14 and a generally u-shaped member 15 secured to a bracket member 16 by means of a pair of mating bolts 17 and nuts 18. The liner assembly 12 comprises a carrying member generally designated by reference number 40 including a thin ring shaped main body portion 41 in use of receptacle 13 is placed into the liner assembly and urine is deposited into it and thereafter sealed with a cap 50.

The Bressler patent is a female urine collection apparatus. A pair of support brackets 33 retain the urine collection device on a toilet bowl rim. The urine collection device which contains a urine collector body 13 is generally in the shape of a parabola. It includes a generally curved opposite side walls 14 which merge into the arcuate front wall 15. They terminate in the bottom wall 17. The side walls and front wall are inclined downwardly and merge into the bottom wall. The bottom wall terminates in a funnel shaped drain 21 an adaptor or cup holder 23 surrounds the funnel shaped drain 21. The adaptor 23 includes a stop portion 27 (see FIG. 3) it limit forward assembly of cup 29 and its flange 31 onto a mounting flange 25 so that the cup is centrally positioned relative to the outlet 19 of the drain 21.

The Middlestead patent is a device which supports the emptying of a colostomy bag 16. It does have a metal wire support which has a first grasping end and a second grasping end in a circular spring formed therebetween to retain the colostomy bag.

The Venter patent discloses a urine sampling device. It is a device which has an elongated trough which is a width substantially greater than its depth to define a generally pan-like trough. The trough is fitted with an attachment to enable it to be attached to the rim of a toilet bowl and it is designed and orientated so that it intercepts a stream of urine being passed by a patient seated on the bowl. The urine sample collecting arrangement comprising a liner for the sampling device the collecting urine being disposed at or towards the extremity of the trough.

The Stingley patent is a more elaborate form of a retaining device which is attached to a toilet bowl for supporting the urine collection cup 200.

The Sapyta patent is a design patent in which a urine cup is held and is supported on the rims of the toilet bowl and has a central opening for retaining the urine specimen cups so that one can urinate into the cup while seated on the toilet bowl.

The House patent is a biological specimen collection apparatus which is a more elaborate form of a collection device which again as you can see from FIG. 4 is supported between the rims of a toilet bowl and has a more elaborate structure to collect urine into the central cup receiving area.

The Paasch patent discloses a urine sample collection device which is a hands free self supporting sample funnel and midstream collecting system for male and female people. The device has support arms 13 that support the device 12 on top of the toilet bowl. A collection section 14 collects urine from the user and funnels the urine towards the opening 18 in a sample cup attachment section 20 for a urine sample cup 16 is attached underneath section 20 for retaining the urine funneled down from collection section 14.

The Paasch Published Patent Application again discloses a urine collecting device which is supported on the rim of a toilet bowl.

The Alvarez patent discloses a device to cover a toilet down and support a urine cup therein.

The El Haje Published Patent Application discloses a urine or stool collection bag.

The House Published Patent Application discloses a device supported on a toilet bowl and which retains a urine collection cup.

The Dijkman Published Patent Application discloses a device for measuring data related to urine production.

There is a significant need for an improved apparatus to enable a patient to provide a urine specimen.

### SUMMARY OF THE INVENTION

The present invention is a hand held apparatus to manually retain a urine specimen cup between the legs of a person so that a person can provide a urine specimen into the cup while precisely holding the cup beneath his or her legs so that the urine can be precisely voided into the cup.

It has been discovered, according to the present invention, that if a winged device includes a hinge at one end and an opening sized to retain a urine specimen cup along the length of the wings, then the wings can be adjusted to fit any desired diameter urine specimen cup and enable the urine specimen cup top be retained between a persons's legs so that a person can precisely urinate into the specimen cup.

It has also been discovered, according to the present invention, that if a device comprises an elongated piece of material with a transverse opening extending therethrough, then a urine specimen cup can be retained in the opening and held between a person's legs so that a person can precisely urinate into the specimen cup.

It has additionally been discovered, according to the present invention, that if a handle having an opening is configured to retain the urine specimen cup and the handle has a wide grip area, then the cup can be easily held in one hand and retained between a persons's legs so that a person can precisely urinate into the specimen cup.

In addition, it has further been discovered that if a breakaway funnel is incorporated into the top of the cup while it is being held, then the urine can be more accurately directed into the cup, especially for a woman.

It is therefore an object of the present invention to provide a winged device which includes a hinge at one end and an opening sized to retain a urine specimen cup along the length of the wings, so that the wings can be adjusted to fit any desired diameter urine specimen cup and enable the urine specimen cup top be retained between a persons's legs so that a person can precisely urinate into the specimen cup.

It is a further object of the present invention t provide a device which comprises an elongated piece of material with a transverse opening extending therethrough, so that a urine specimen cup can be retained in the opening and held between a person's legs so that a person can precisely urinate into the specimen cup.

It is an additional object of the present invention to create a handle having an opening configured to retain the urine specimen cup and further design a wide grip area into the handle, so that the cup can be easily held in one hand and retained between a persons's legs so that a person can precisely urinate into the specimen cup.

It is also another object of the present invention to incorporate a breakaway funnel into the top of the cup so that the urine can be more accurately directed into the cup, especially for a woman.

Further novel features and other objects of the present invention will become apparent from the following detailed description, discussion and the appended claims, taken in conjunction with the drawings.

### BRIEF DESCRIPTION OF THE DRAWINGS

Referring particularly to the drawings for the purpose of illustration only and not limitation, there is illustrated:

FIG. 1 is a top plan view of a first embodiment of the present invention urine cup specimen holder;

FIG. 2 is a side elevational view of the first embodiment of the present invention illustrated in FIG. 1 retaining a urine specimen cup;

FIG. 3 is a top plan view of a second embodiment of the present invention urine cup specimen holder;

FIG. 4 is a side elevational view of the second embodiment of the present invention illustrated in FIG. 3 retaining a urine specimen cup;

FIG. 5 is a side elevational view of a third embodiment of the present invention urine specimen cup holder; and

FIG. 6 is a side elevational view of an addition of a breakaway funnel into the urine specimen cup holder illustrated in FIG. 5.

### DETAILED DESCRIPTION OF THE PREFERRED EMBODIMENTS

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

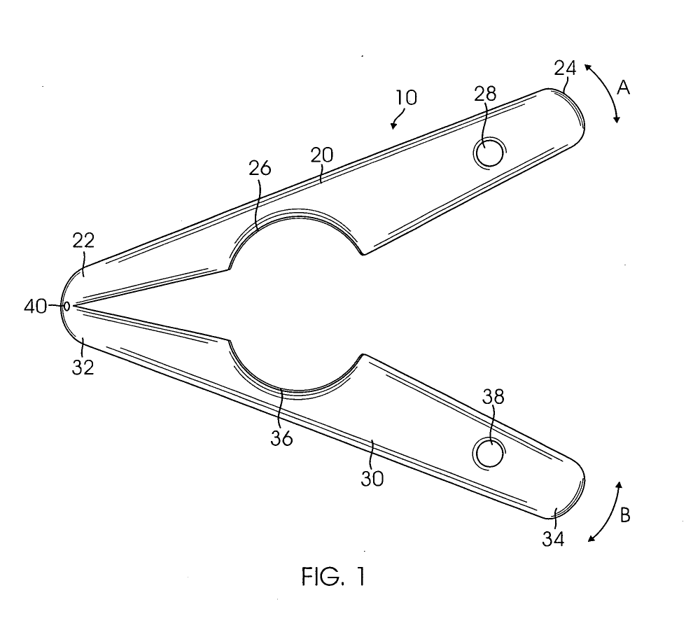

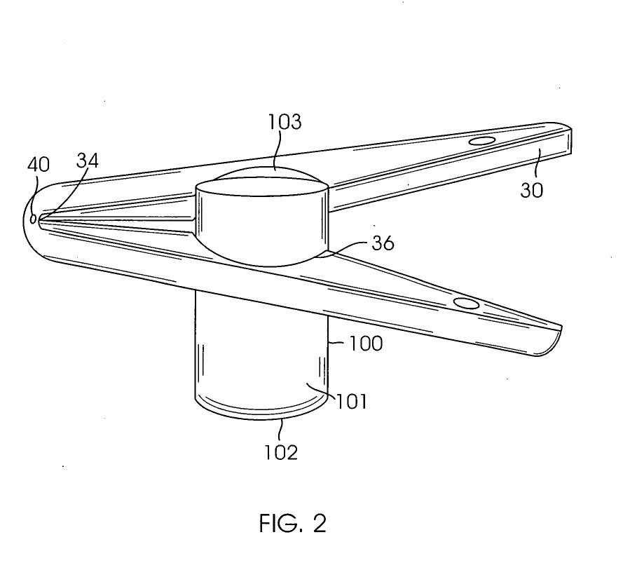

Referring to FIGS. 1 and 2, there is illustrated a first embodiment of the present invention urine specimen cup holder 10. The holder 10 is a winged device having a first wing member 20 and a second wing member 30. The first wing member 20 has a leading end 22 and a trailing end 24. The second wing member 30 has a leading end 32 and a trailing end 34. The leading ends 22 and 32 are rotatably joined by a rotatable member 40 which may be a simple bolt extending through the leading end and about which the leading ends can rotate or a hinge about which the leading ends 22 and 32 can rotate. Wing 20 can rotate in the direction of arrows “A” and wing 30 can rotate in the direction of arrows “B”. Wing 20 has a transverse curved opening 26 extending through its entire thickness “T” and wing 30 has a transverse curved opening 36 extending through its entire thickness “T”. When the wings 20 and 30 are rotated toward each other so that their transverse curved openings 26 and 36 come together, a urine specimen cup 100 is retained between the wings 20 and 36 at the location of the transverse curved openings. The curved openings of each wing at least partially conform to the cylindrical shape of a urine specimen cup 100. Because the wings 20 and 30 can be rotated apart, the device 10 can accommodate urine specimen cups of different diameters. Optionally, each wing 20 and 30 can retain a finger detent 28 and 38 to facilitate grasping the device.

The urine specimen cup has a cylindrical sidewall 101 and a bottom 102 which form an interior chamber 102 into which urine is deposited.

In use, the user places a urine specimen cup at the location of the transverse curved openings 26 and 36 and closes the wings 20 and 30 so that the urine specimen cup 100 is retained between the wings 20 and 30 and then user holds the specimen cup holder 10 in one hand or both hands and places it between his or her legs so that urine can be precisely voided into the urine specimen cup.

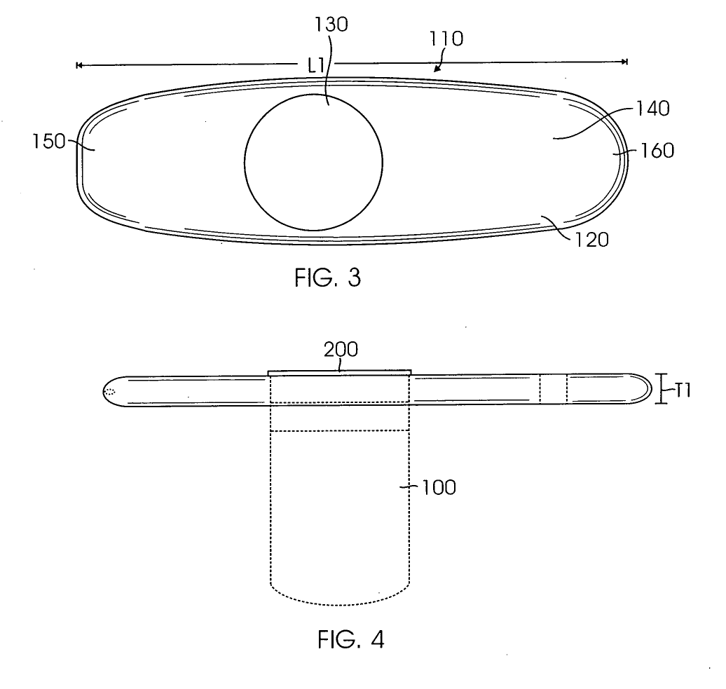

Referring to FIGS. 3 and 4, there is illustrated a second embodiment of the present invention urine specimen cup holder 110. The holder 110 is comprised of an elongated member body 120 of a given length “L1” and a given thickness “T1”. The body 120 has a transverse opening 130 which preferably is circular and extends through the entire thickness “T1” of the body 120. The opening 130 can be located at the longitudinal center or can be offset from the longitudinal center as illustrated in FIG. 3. The diameter “D1” of the opening 130 is smaller than the rim 200 of urine specimen cup 100 so that the cup is retained on the upper surface of the body 120.

In use, a user places a urine specimen cup into the opening 130 and holds the body 120 in one hand at any location on the body but preferably adjacent the first end 150 or the second end 160 and places the body 120 between his or her legs so that urine can be precisely voided into the urine specimen cup.

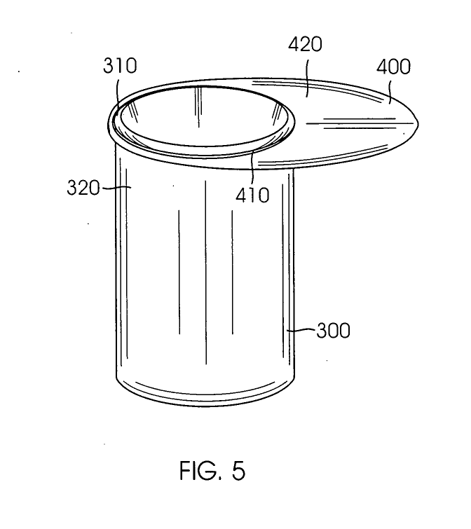

Referring to FIG. 5, there is illustrated a third embodiment of the present invention urine cup holder. The third embodiment involves incorporating a handle 400 which has an opening 410 to receive the urine specimen cup 300 which has a rim 310 which is slightly larger than the diameter of opening 410. As part of this embodiment, the handle 410 can be incorporated into and be a part of the sidewall 320 of the urine specimen cup 300. Therefore, the handle 400 can be molded as an integral part of the urine specimen cup 300. As an additional feature, the handle 400 can have an extra wide surface area 420 which extends for nearly half the circumference of the rim 310 of the cup 300 so that the handle is easier to grip.

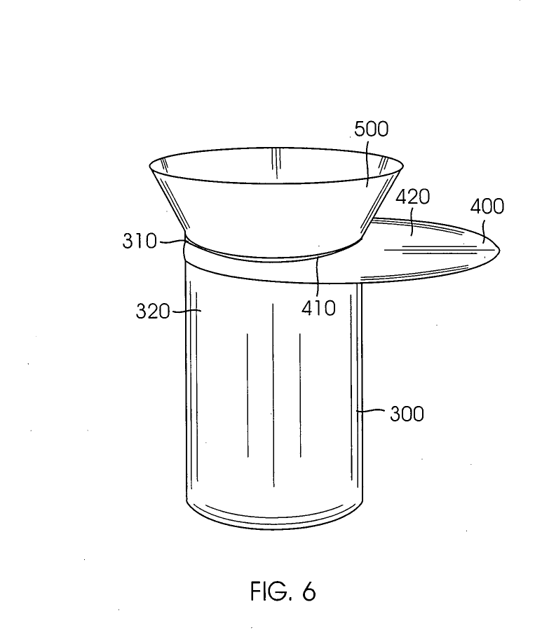

Referring to FIG. 6, there is incorporated as an additional feature a funnel 500 which is incorporated into the rim 310 of the cup 300. The funnel 500 can have a breakaway design so that it can be removed from the cup after the urination process is completed. The funnel 400 helps to direct the flow of urine into the cup. This is especially beneficial for women.

For the embodiments illustrated in FIGS. 5 and 6, in use, the user holds the urine specimen cup 300 in one hand by the handle 400 and places the urine specimen cup 300 between his or her legs so that urine an be precisely voided into the urine specimen cup.

For each of the embodiments, the urine specimen cup holder can be made out of any desired material such as plastic, polyurethane, wood or even hard cardboard.

The present invention is defined as a urine specimen cup holder wherein the urine specimen cup has a cylindrical sidewall and a bottom wall which surround an interior chamber and a top opening leading to the chamber, the urine specimen cup holder comprising: (a) a first elongated body member having a front end, a rear end, a top surface, a given thickness, and a transverse curved opening at a location long one side of the first elongated body and extending through the thickness of the body; (b) a second elongated body member having a front end, a rear end, a top surface, a given thickness, and a transverse curved opening at a location long one side of the second elongated body and extending through the thickness of the body; and (c) the first and second elongated body rotatably connected to each other at a location adjacent their respective first end so that their respective transverse curved opening face each other and are aligned with each other; (d) whereby the cylindrical sidewall of the urine specimen cup is retained between the first and second elongated body at the location of the aligned transverse curved openings so that the top opening of the urine specimen cup holder rests adjacent to the top surface of the first and second elongated body.

The present invention is defined alternatively as a urine specimen cup holder wherein the urine specimen cup has a cylindrical sidewall and a bottom wall which surround an interior chamber and a top opening leading to the chamber and a rim surrounding the top opening, the urine specimen cup holder comprising: (a) an elongated member body of a given length and a given thickness, the elongated member body having an upper surface and a transverse opening extending through the entire thickness of the body and sized to receive the cylindrical sidewall of the urine specimen cup so that the rim of the urine specimen cup rests on the top surface of the body.

The present invention is also defined alternatively as a urine specimen cup holder wherein the urine specimen cup has a cylindrical sidewall and a bottom wall which surround an interior chamber and a top opening surrounded by rim leading to the chamber, the urine specimen cup holder comprising a handle having an opening to receive the urine specimen cup so that the rim rests on the handle, the handle having a wide surface area to facilitate gripping.

Of course the present invention is not intended to be restricted to any particular form or arrangement, or any specific embodiment, or any specific use, disclosed herein, since the same may be modified in various particulars or relations without departing from the spirit or scope of the claimed invention hereinabove shown and described of which the apparatus or method shown is intended only for illustration and disclosure of an operative embodiment and not to show all of the various forms or modifications in which this invention might be embodied or operated.

What is claimed is:

1. A urine specimen cup holder wherein the urine specimen cup has a cylindrical sidewall and a bottom wall which surround an interior chamber and a top opening leading to the chamber, the urine specimen cup holder comprising:
a. a first elongated body member having a front end, a rear end, a top surface, a given thickness, and a transverse curved opening at a location long one side of the first elongated body and extending through the thickness of the body;
b. a second elongated body member having a front end, a rear end, a top surface, a given thickness, and a transverse curved opening at a location long one side of the second elongated body and extending through the thickness of the body; and
c. the first and second elongated body rotatably connected to each other at a location adjacent their respective first end so that their respective transverse curved opening face each other and are aligned with each other;
d. whereby the cylindrical sidewall of the urine specimen cup is retained between the first and second elongated body at the location of the aligned transverse curved openings so that the top opening of the urine specimen cup holder rests adjacent to the top surface of the first and second elongated body.

2. The urine specimen cup holder in accordance with claim 1 where the first elongated body and the second elongated body are made from material selected from the group consisting of plastic, polyurethane, wood and cardboard.

3. The urine specimen cup holder in accordance with claim 1 further comprising a finger detent in the top surface of the first elongated member and a finger detent in the top surface of the second elongated member.

4. A urine specimen cup holder wherein the urine specimen cup has a cylindrical sidewall and a bottom wall which surround an interior chamber and a top opening leading to the chamber and a rim surrounding the top opening, the urine specimen cup holder comprising:
a. an elongated member body of a given length and a given thickness, the elongated member body having an upper surface and a transverse opening extending through the entire thickness of the body and sized to receive the cylindrical sidewall of the urine specimen cup so that the rim of the urine specimen cup rests on the top surface of the body.

5. The urine specimen cup holder in accordance with claim 4 wherein the transverse opening is positioned at the longitudinal center of the elongated member body.

6. The urine specimen cup holder in accordance with claim 4 wherein the transverse opening is positioned offset from the longitudinal center of the elongated member body.

7. The urine specimen cup holder in accordance with claim 4 where the first elongated body and the second elongated body are made from material selected from the group consisting of plastic, polyurethane, wood and cardboard.

8. A urine specimen cup holder wherein the urine specimen cup has a cylindrical sidewall and a bottom wall which surround an interior chamber and a top opening surrounded by rim leading to the chamber, the urine specimen cup holder comprising:
a. a handle having an opening to receive the urine specimen cup so that the rim rests on the handle, the handle having a wide surface area to facilitate gripping.

9. The urine specimen cup in accordance with claim 8 wherein the handle is formed into the sidewall of the urine specimen cup.

10. The urine specimen cup in accordance with claim 8 further comprising a funnel formed into the rim of the urine specimen cup.

11. The urine specimen cup in accordance with claim 10 wherein the funnel is a breakaway funnel which permits the funnel to be removed from the rim.
